(19) United States
(12) Patent Application Publication
(10) Pub. No.: US 2005/0186670 A1
(43) Pub. Date: Aug. 25, 2005

(54) Method of detecting error spot in DNA chip and system using the method

(76) Inventor: Ji-young Oh, Suwon-si (KR)

Correspondence Address: CANTOR COLBURN, LLP, 55 GRIFFIN ROAD SOUTH, BLOOMFIELD, CT, 06002, US

(21) Appl. No.: 11/061,018

(22) Filed: Feb. 18, 2005

(30) Foreign Application Priority Data
Feb. 21, 2004 (KR) 10-2004-0011654

(51) Int. Cl. G11B 7/00; G01B 11/26; C12M 1/34

(52) U.S. Cl. 435/287.2; 369/53.13; 356/139.04

(57) ABSTRACT

Provided are a method of detecting an error spot and a system using the method. The method includes analyzing a difference in variances of a background intensity and a foreground intensity for each spot in a DNA chip, verifying whether the mean of the background intensity and the mean of the foreground intensity are significantly different from each other based on the difference in variances, and judging an error spot based on the results of the verifying operation. Thus, the reliability in statistical analysis can be increased by excluding the error spot in the statistical analysis.

### BACKGROUND OF THE INVENTION

This application claims the benefit of Korean Patent Application No. 10-2004-0011654, filed on Feb. 21, 2004, in the Korean Intellectual Property Office, the disclosure of which is incorporated herein in its entirety by reference.

1. Field of the Invention

The present invention relates to a method of detecting an error spot and a system using the method, and more specifically, to a method of detecting an error spot by quantifying DNA chips and a system using the method.

2. Description of the Related Art

DNA chips have been manufactured using molecular biological technologies and newly developed mechanical and electronic engineering technologies. DNA chips are chips in which several hundreds to several hundreds of thousands DNAs are integrated in a very small space using mechanical automation and electronic control technologies. That is, DNA chips are chips to which many types of DNAs are attached with high density for detecting genes. DNA chips can replace the conventional genetic engineering technologies, such as southern blotting and northern blotting, mutant detection, and DNA sequencing.

DNA chips are classified into four groups depending on the manufacturing method; pin microarray chips manufactured by micro dotting (surface contact) using a pin, inkjet chips manufactured by micro dotting using an inkjet technology, photolithography chips, and electronic array chips.

FIG. 1 is a flowchart illustrating a conventional method of analyzing genes using a DNA chip.

Referring to FIG. 1, a sample preparation is performed for taking a sample, i.e., gene to be analyzed (operation (S100)). In the sample preparation, pure genes to be analyzed are extracted from a biological sample, such as blood.

Next, genes extracted via the sample preparation are amplified to an analyzable level (operation (S110)). The amplification operation is generally performed by a polymerase chain reaction (PCR).

Then, the amplified genes, which are a target sample, are hybridized in the DNA chip (operation (S120)). In the hybridization operation, the target sample to be tested is reacted with oligo samples having information of genes and immobilized on the chip. Thus the target sample is hybridized with an oligo sample having a complementary sequence.

Next, a non-hybridized target sample which remains on the chip is washed off (operation (S130)). Then, the image of the chip is scanned by a scanner to detect a degree of hybridization of the target sample with the oligo probe (operation (S140)). Subsequently, the scanned image is quantified for a statistical analysis (operation (S150)).

After quantifying the image of the DNA chip, a statistical analysis is performed using various algorithms and the quantified value of each spot on the chip is analyzed in order to discriminate whether the target sample is originated from a sick person or a normal person (operation (S160)).

As illustrated in FIG. 1, the conventional method of analyzing genes comprises a series of continuous seven operations. During experiments between the first operation and the fifth operation (operations (S100 through S140)), various error factors and thus various types of error spots are generated. If the quantification operation is performed based on false information due to the errors and the statistical analysis is performed using the quantified false data, the false spot data may reduce a reliability of the analysis and limit the possibility to identify a sick person.

### SUMMARY OF THE INVENTION

The present invention provides a method of detecting an error spot, which increases a reliability in a statistical analysis by detecting the error spot in a DNA chip and excluding the detected error spot in the statistical analysis and a system using the method.

The present invention also provides a computer-readable recording medium having recorded therein a computer program for executing in a computer a method of detecting an error spot, the method increasing a reliability in a statistical analysis by detecting the error spot in a DNA chip and excluding the detected error spot in the statistical analysis.

According to an aspect of the present invention, there is provided a method of detecting an error spot, comprising the operations of: analyzing a difference in variances for a background intensity and a foreground intensity for each spot in a DNA chip; verifying if a mean of the background intensity and a mean of the foreground intensity are significantly different from each other, based on the difference in variances; and judging an error spot based on the results of the verifying operation.

According to another aspect of the present invention, there is provided a system for detecting an error spot, comprising: a variance analysis part for analyzing a difference in variances for background intensity and a foreground intensity for each spot in a DNA chip; a mean verifying part for verifying whether a mean of the background intensity and a mean of the foreground intensity are significantly different from each other, based on the difference in variances; and an error spot judging part for judging an error spot based on the results of the verifying operation.

According to still another aspect of the present invention, there is provided a computer-readable recording medium having recorded thereto a computer program for executing in a computer a method of detecting an error spot, the method comprising the operations of: analyzing a difference in variances for a background intensity and a foreground intensity for each spot in a DNA chip; verifying whether a mean of the background intensity and a mean of the foreground intensity are significantly different from each other based on the difference in variances; and judging an error spot based on the results of the verifying operation.

### BRIEF DESCRIPTION OF THE DRAWINGS

The above and other features and advantages of the present invention will become more apparent by describing in detail exemplary embodiments thereof with reference to the attached drawings in which:

FIG. 1 is a flowchart illustrating a conventional method of analyzing genes using a DNA chip;

FIG. 2 is a flowchart illustrating an image processing procedure for a DNA chip;

FIG. 3 is a diagram illustrating an image scanning of a DNA chip;

FIG. 4 is a diagram illustrating errors generated during analyzing a DNA chip and the types of scanning errors corresponding to the errors generated during analyzing the DNA chip;

FIG. 5 is a diagram illustrating results generated from the types of scanning errors in FIG. 4;

FIG. 6A is a graph illustrating the relationship between a spot size and a spot intensity;

FIG. 6B is a graph illustrating the relationship between a spot intensity and its standard deviation;

FIGS. 7A and 7B are diagrams illustrating input data used in a method of detecting an error spot according to an embodiment of the present invention;

FIG. 8 is a flowchart illustrating a method of detecting an error spot according to an embodiment of the present invention;

FIG. 9 is a block diagram illustrating a system for detecting an error spot according to another embodiment of the present invention;

FIGS. 10 and 11 are diagrams illustrating the ratio and the type of error spots detected in each DNA chip; and

FIG. 12 is a diagram illustrating a change of Robust M caused by excluding error spots.

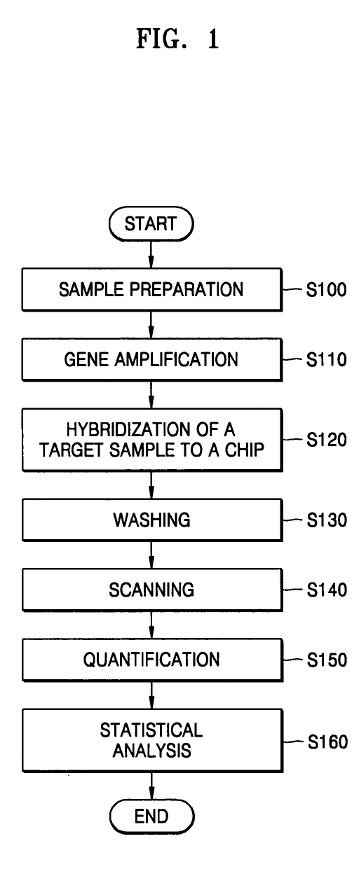

### DETAILED DESCRIPTION OF THE INVENTION

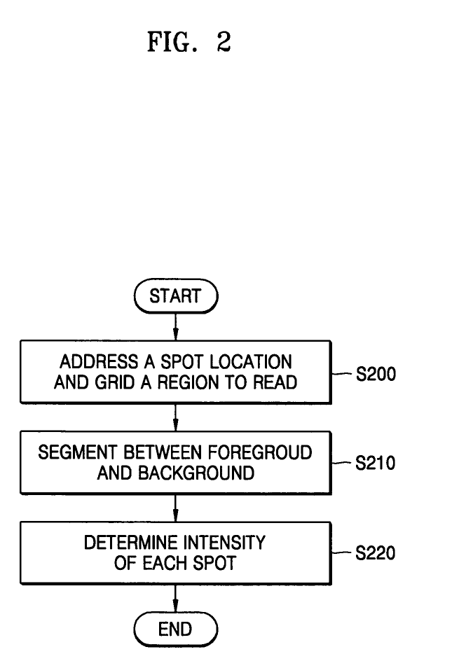

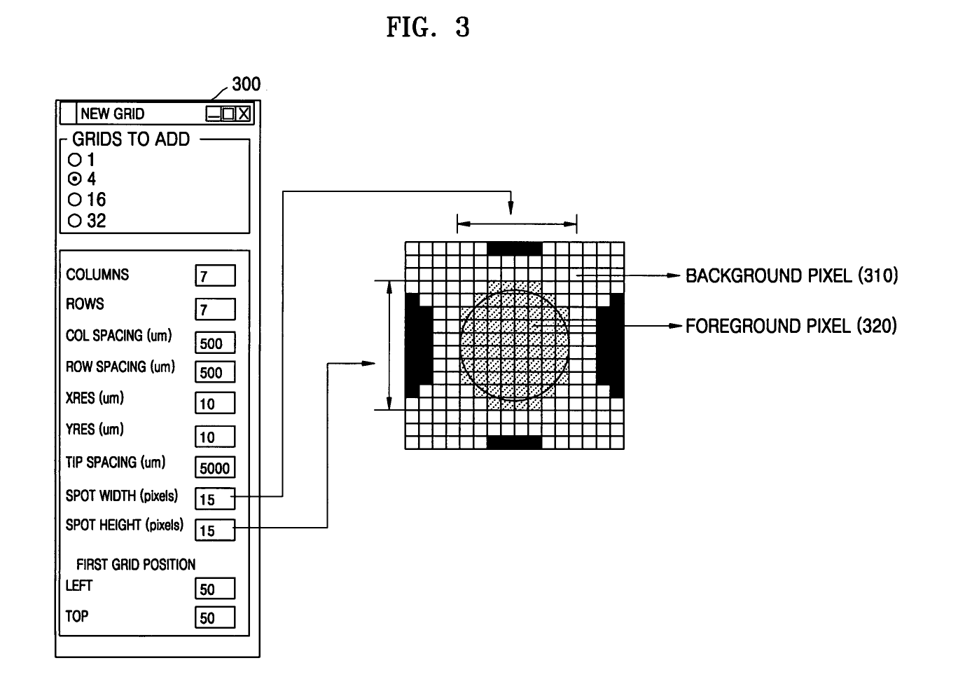

FIG. 2 is a flowchart illustrating an image processing procedure for a DNA chip and FIG. 3 is a diagram illustrating an image scanning of a DNA chip.

In general, the image processing procedure of a DNA chip includes a scanning operation and a quantification operation. The scanning operation and the quantification operation are closely related to each other. Values obtained from the quantification operation change depending on a scanning method.

Referring to FIGS. 2 and 3, there is first performed addressing of a location and a shape of each spot in the DNA chip and gridding of a region to be read (operation (S200)).

Next, a segmentation is performed (operation (210)) in which pixels belonging to a background region (310) and pixels belonging to a foreground region (320) in the addressed spot are segmented. Various methods have been proposed to segment the foreground (320) and the background (310). Representative methods include a fixed circle assumption and an adaptive circle assumption.

The fixed circle assumption method segments a background and a foreground by plotting identical circles for each spot, on the assumption that all spots have the same size and shape. The adaptive circle assumption plots a shape of a spot by connecting pixels having an intensity remarkably different from adjacent pixels, by taking it into account that each spot may have a different shape and a different size.

After segmenting the background and the foreground (operation (S210)), a median value of the intensity is read for each pixel in the background and the foreground, respectively, and the median values are summed up and then divided by the number of pixels to obtain a mean of the intensity for the background and the foreground, respectively. In addition, a standard deviation for the background and the foreground, respectively, is obtained based on the median values of the intensity for each pixel.

Also, various methods of quantifying an intensity by scanning the spot are disclosed. Representative quantifying methods include a method using a standard deviation of a background, a method using a spotted area, and a method using a center point.

The method using a standard deviation of a background is performed based on the percentage of pixels in a foreground, a median intensity of each pixel, which is larger than a median intensity for a background, being added to one or two times its standard deviation. This method is sensitive to the standard deviation of the intensity. However, it is difficult to determine a critical value of the percentage and discriminate an error in alignment and a spot shape.

The method using a spotted area discriminates an error spot by comparing the area of a foreground with the area of gridded region in the spot.
Spot shape QC score=(spot area=pR²/2pR)/(spot perimeter=R/2)

If QC score≦R/2, the spot is regarded as an error spot.

That is, as a result of the above comparison of the areas, if the area of a foreground is less than R/2, the spot is regarded as an error spot. However, this method cannot distinguish the errors, such as intensity error, spot spreading, non-uniformity of a background and the like.

The method using the center point of a spot comprises comparing the differences between the center point of a spot which was gridded in an immobilized state and the center point of a spot which was gridded in a flexible state and classifying spots having a considerable difference as error spots. However, this method cannot distinguish the errors, such as intensity error, spot spreading and the like.

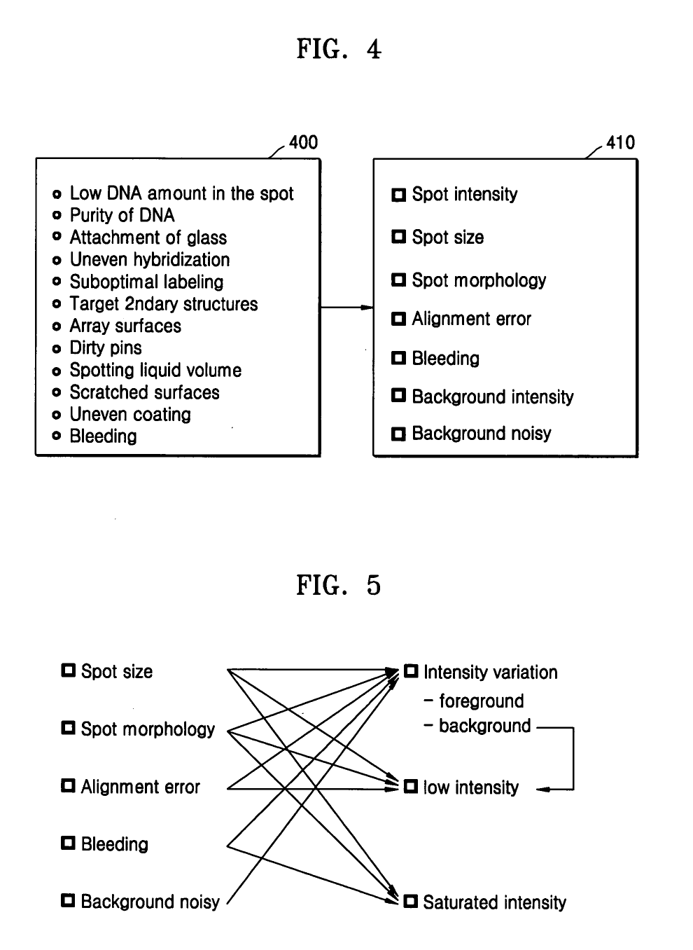

FIG. 4 is a diagram illustrating errors generated during analyzing a DNA chip and the types of scanning errors corresponding to the errors generated during analyzing the DNA chip.

Referring to FIG. 4, the errors (400) generated during analyzing a DNA chip include (1) low DNA amount in the spot, (2) purity of DNA, (3) attachment of glass, (4) uneven hybridization, (5) suboptimal labeling, (6) target 2ndary structures, (7) array surfaces, (8) dirty pins, (9) spotting liquid volume, (10) scratched surfaces, (11) uneven coating, (12) bleeding and the like.

The types of the corresponding scanning errors generated from the errors (400) include (1) spot intensity, (2) spot size, (3) spot morphology, (4) alignment error, (5) bleeding, (6) background intensity, (7) background noisy and the like.

FIG. 5 is a diagram illustrating results resulting from the types of scanning errors as illustrated in FIG. 4.

Referring to FIG. 5, intensity variation results from the errors of spot size, spot morphology, alignment error, bleeding, and background noisy. Low intensity results from the errors of spot size, spot morphology, alignment error, and bleeding. Further, saturated intensity results from the errors of spot size, spot morphology, and bleeding.

Thus, as a result of analyzing the relationship between the error types in the DNA chip and results thereof, the error types are classified as spots exhibiting (1) low intensity, (2) intensity variation in the foreground and the background, or (3) saturated intensity.

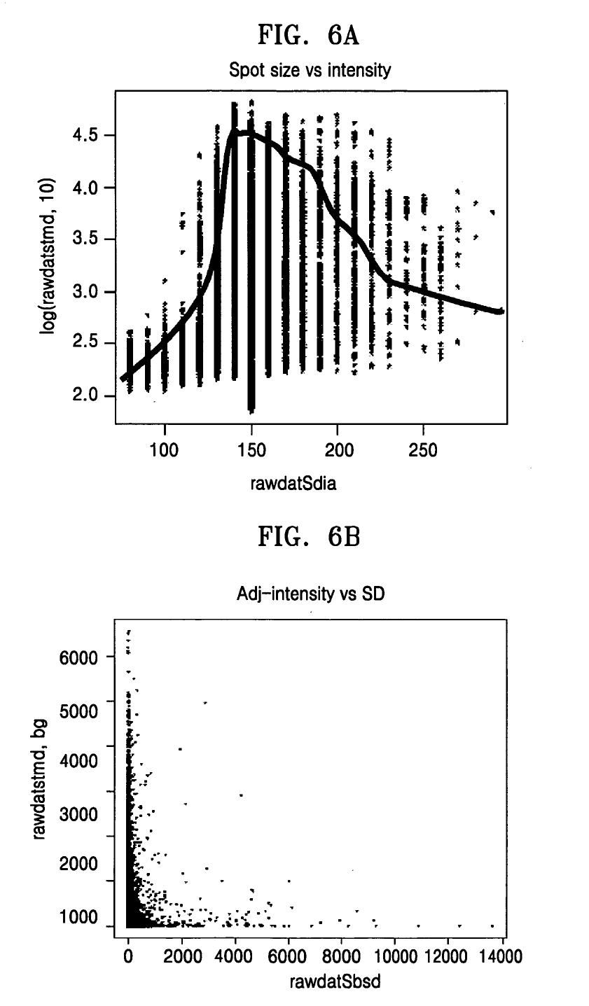

FIG. 6A is a graph illustrating the relationship between a spot size and a spot intensity, and FIG. 6B is a graph illustrating the relationship between a spot intensity and its standard deviation.

Referring to FIG. 6B, the statistical result is that as the deviation of the intensity is higher, a possibility that the intensity is low is higher.

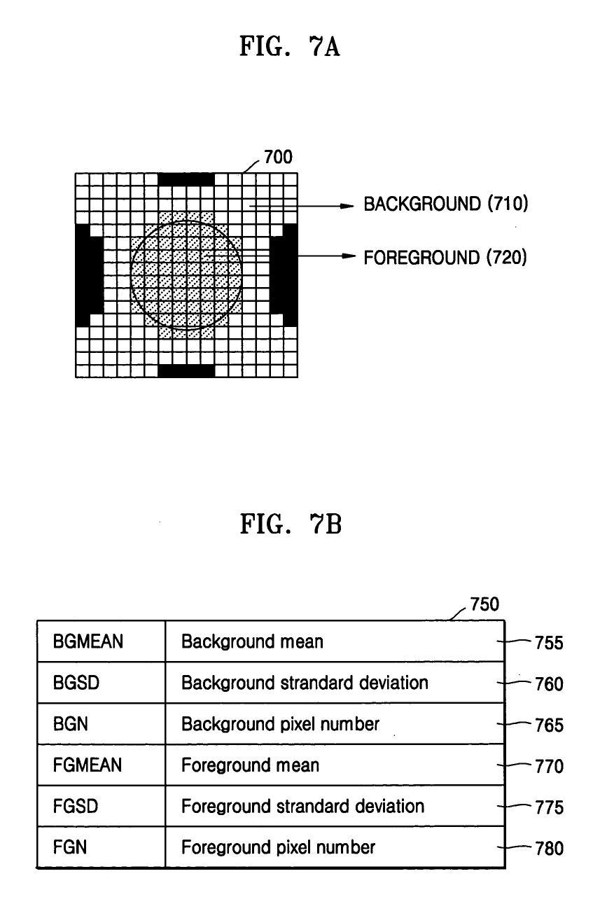

FIGS. 7A and 7B are diagrams illustrating examples of input data used in a method of detecting an error spot according to an embodiment of the present invention.

Referring to FIGS. 7A and 7B, the spots (700) are segmented into the foreground (720) and the background (710). Then, a foreground mean (770) is obtained by dividing a median of intensity of each pixel comprising the foreground (720) by the foreground pixel number (780). And a foreground standard deviation (775) is obtained from the foreground mean (770). Also, a background mean (775) is obtained by dividing a median of intensity of each pixel comprising the background (710) by the background pixel number (765). And a background standard deviation (760) is obtained from the background mean (775).

Thus, input data (750) used in a method of detecting an error spot according to an embodiment of the present invention consist of the mean (770) and the standard deviation (775) for the foreground intensity and the foreground pixel number (780), and the mean (755) and the standard deviation (760) for the background intensity and the background pixel number (765).

There are many programs for quantifying the spot intensity of the DNA chip, each program showing a mean, standard deviation, and pixel number for the background and the foreground, respectively, as a result of quantification. Thus, if the quantification is possible using a conventional program, variables necessary to perform an embodiment of the present invention may be extracted from the files output as a result of the quantification. In general, the quantification program outputs files with a GPR file extension.

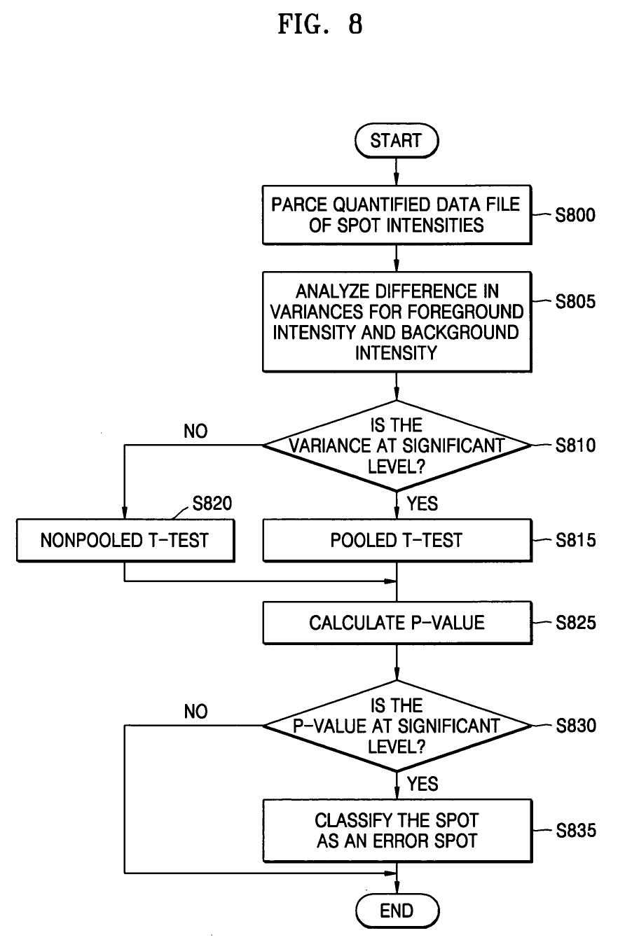

FIG. 8 is a flowchart illustrating a method of detecting an error spot according to an embodiment of the present invention.

Referring to FIG. 8, the quantification program produces an output file including the respective mean, standard deviation, and pixel number for foreground intensity and background intensity of the spot. A conventional quantification program can be used in the embodiment of the present invention.

The output file is subject to parcing (operation (S800)), in order to extract input data consisting of the respective mean, standard deviation, and pixel number for foreground intensity and background intensity of the spot, which are necessary to the present invention from the output file.

Then, the difference in variances is analyzed using the standard deviations for each foreground intensity and background intensity, respectively ((operation (S805)). The f-test is used for analyzing the difference in variances. The f-test is used to verify whether variances of two groups are significantly different from each other.

After the completion of the analysis ((operation (S805)), a verifying operation is performed to establish whether the mean of the background intensity and the mean of the foreground intensity are significantly different from each other, based on the difference in variances ((operations (S810 through S815)). If the results of the difference in variances obtained from the f-test are significant, a pooled t-test is performed for verifying the means. Contrary to this, if the results of the difference in variances obtained from the f-test are not significant, a non-pooled t-test is performed for verifying the means. For example, the resulting value of at least 0.05 in the f-test is judged as being significant, and the resulting value of no more than 0.05 in the f-test is judged as not being significant. The value of 0.05, which is used as a criterion for the establishing the significance, can be somewhat changed depending on the results of statistical results.

The t-test is used to verify whether means of two groups are significantly different or not.

$$Equation 1$$

$$test statistic t = \frac{( {\overline{Y}}_{1} - {\overline{Y}}_{2} ) - ( {μ}_{γ1} - {μ}_{γ2} )}{{S}_{γ1 - γ2}} = \frac{{\overline{Y}}_{1} - {\overline{Y}}_{2}}{{S}_{γ1 - γ2}} , \\ wherein , {H}_{0} : {μ}_{γ1} - {μ}_{γ2} = 0 \qquad ( 1 )$$

In equation 1, t represents a difference between the means (μ_{γ1}, μ_{γ2}) of the two groups in a pooled t-test, which is used when the two groups have a similar type of deviation.

$$\begin{matrix}Equation 2 & \\ t = \frac{( {\overline{X}}_{1} - {\overline{X}}_{2} ) - ( {μ}_{1} - {μ}_{2} )}{\sqrt{\frac{{( {S}_{1} )}^{2}}{{n}_{1}} + \frac{{( {S}_{2} )}^{2}}{{n}_{2}}}} & ( 2 ) \\ Equation 3 & \\ degree of freedom ( df ) = \frac{[ {( {S}_{1} )}^{2} / {n}_{1} + {( {S}_{2} )}^{2} / {n}_{2} ]}{[ ( {( {S}_{1} )}^{2} / ( {n}_{1} - 1 ) ) + ( {( {S}_{2} )}^{2} / ( {n}_{2} - 1 ) ) ]} & ( 3 )\end{matrix}$$

In equation 2, t represents a significant difference between the means of the two groups in a non-pooled t-test, and in equation 3, df represents degrees of freedom. If a variance between the two groups is high, the degrees of freedom are increased, and then the difference between the means is analyzed. Thus, the significant difference between the means is affected by the difference in variances.

After performing the pooled t-test or the non-pooled t-test depending on the difference in variances, a p-value is calculated, based on the result of the pooled or non-pooled t-test (operation (S825). If the p-value is at a significant level, the detected spot is judged as an error spot (operation (S835)). For example, if the p-value is at least 0.05, the p-value is judged as being at significant level and the detected spot is classified as an error spot. The value of 0.05, which is used as a criterion for the judgment of the significance level, can be somewhat changed depending on the results of statistical experimental results.

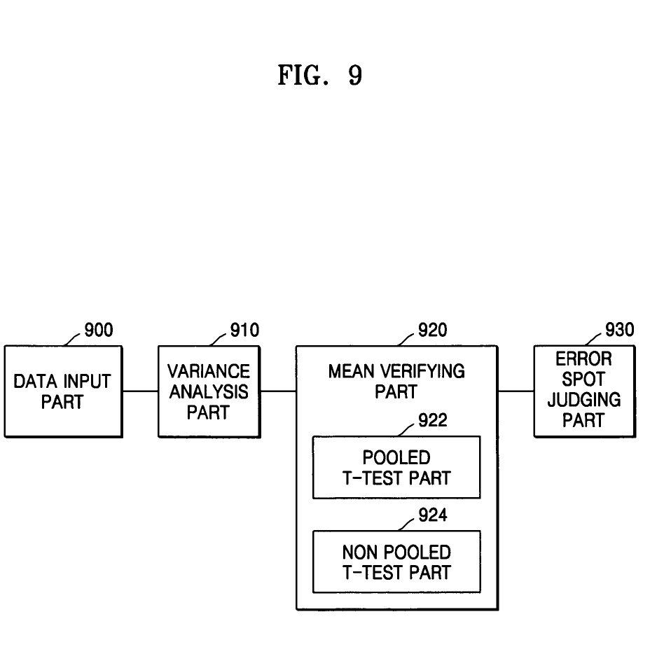

FIG. 9 is a block diagram illustrating a system for detecting an error spot according to an embodiment of the present invention.

Referring to FIG. 9, the system for detecting an error spot is composed of a data input part (900), a variance analysis part (910), a mean verifying part (920) and an error spot judging part (930). The mean verifying part (920) is composed of a pooled t-test part (922) and a non-pooled t-test part (924) operating corresponding to the difference in variances.

The data input part (900) receives a file including the results of the quantification operation. In addition, the data input part (900) extracts input data which are necessary to detect an error spot, from the file. In an embodiment of the present invention, since analyzing variances and verifying means are performed to detect the error spot, the respective mean, standard deviation, and pixel number for background intensity and foreground intensity are extracted to obtain the input data from the file.

In the variance analysis part (910), analysis of the difference in variances for the background intensity and the foreground intensity is performed based on a standard deviation of the input data extracted in the data input part (900). The analysis of the variance is performed using the f-test.

In the mean verifying part (920), verification is performed whether the mean of the background intensity and the mean of the foreground intensity are significantly different from each other, based on the difference in variances in the variance analysis part (910). The verification is performed using the t-test. The variance analysis part (920) may perform the pooled t-test in a pooled t-test part (922) or the non-pooled t-test in a non-pooled t-test part (924), depending the difference in variances.

For example, if the resulting value in the f-test is at least 0.05, the difference in variances are judged as having a significance, and the non-pooled t-test is performed. If the resulting value in the f-test is no more than 0.05, the pooled t-test is performed.

In the error spot judging part (930), the p-value is calculated based on the results in the mean verifying part (920) and a judgment on an error spot is performed based on the p-value. For example, if the p-value is at least 0.05, the detected spot is classified as an error spot.

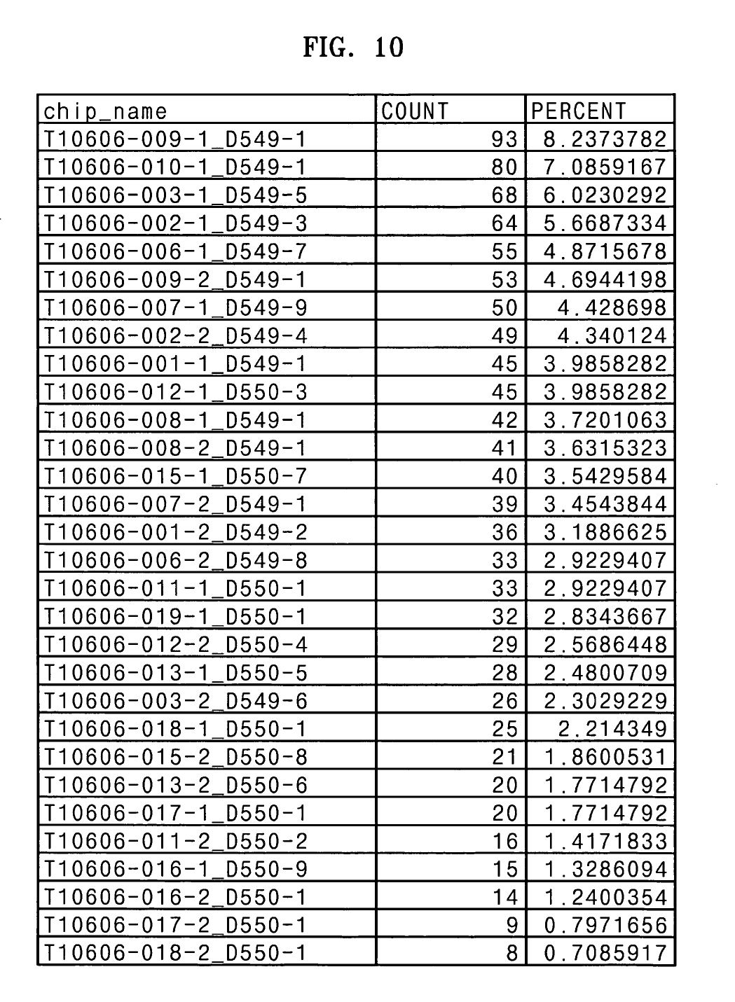

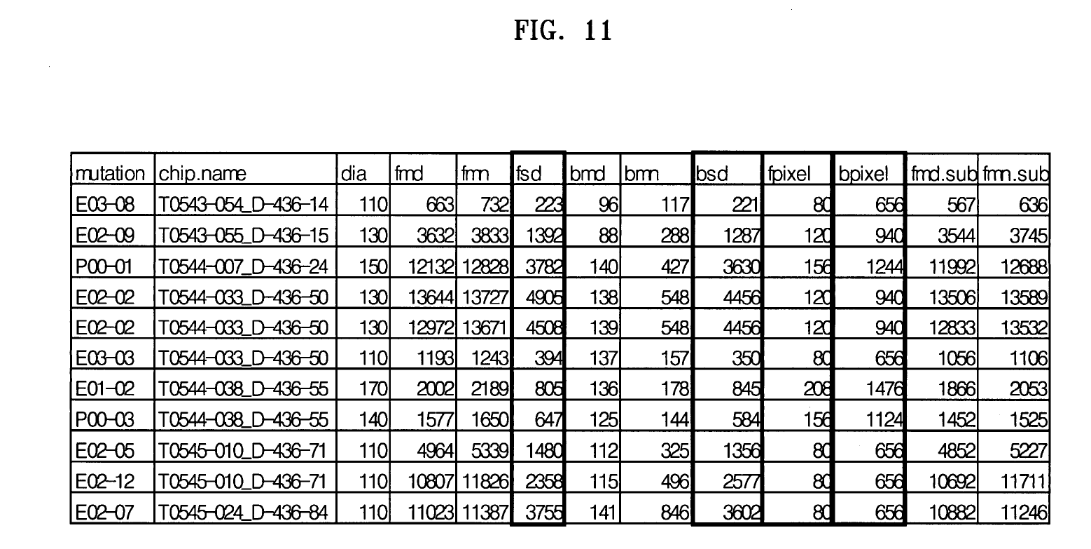

FIGS. 10 and 11 are diagrams illustrating the ratio and the type of error spots detected in each DNA chip.

Referring to FIG. 10, 0.7 to 8.23% of the spots are detected as error spots. As a result of analyzing the data detected as error spots, while in most error spots, the standard deviation of the foreground intensity (fsd) and the standard deviation of the background intensity (bsd) are high and the foreground intensity (fmd) and the background intensity (bmd) are low, some spots having a high standard deviation of the intensity may be detected as error spots even though their intensities are more than 10000.

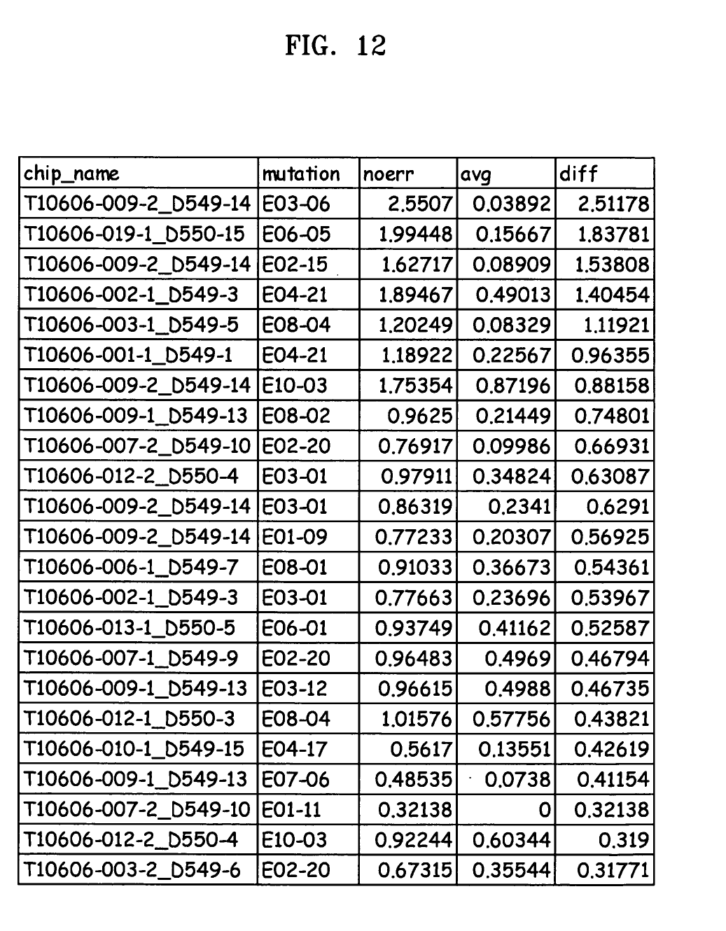

FIG. 12 is a diagram illustrating a change of Robust M caused by excluding error spots.

Referring to FIG. 12, with respect to the change of Robust M, the difference is no more than about 2.5. This is a great difference, taking it into account that if the difference is at least 1 in the analysis, the kernel discriminating the difference changes greatly. Thus, reliability on the results may be increased.

The invention can also be embodied as computer readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, and carrier waves (such as data transmission through the Internet). The computer readable recording medium can also be distributed over network coupled computer systems so that the computer readable code is stored and executed in a distributed fashion.

According to an embodiment of the present invention, spots having high difference in variances for the foreground intensity and the background intensity are detected as error spots (such as, spots having low intensity resulting from small spot size or incorrect alignment, or spots having partially saturated intensity) and excluded, and thus in the subsequent statistical analysis, errors in discriminating between a sample from a normal person and a sample from a patient can be decreased. That is to say, the reliability in statistical analysis can be increased.

While this invention has been particularly shown and described with reference to preferred embodiments thereof, it will be understood by those skilled in the art that various changes in form and details may be made therein without departing from the spirit and scope of the invention as defined by the appended claims. The preferred embodiments should be considered in descriptive sense only and not for purposes of limitation. Therefore, the scope of the invention is defined not by the detailed description of the invention but by the appended claims, and all differences within the scope will be construed as being included in the present invention.

1. A method of detecting an error spot, comprising the operations of:
analyzing a difference in variances for a background intensity and a foreground intensity for each spot in a DNA chip;
verifying if a mean of the background intensity and a mean of the foreground intensity are significantly different from each other, based on the difference in variances; and
judging an error spot based on the results of the verifying operation.

2. The method of claim 1, wherein the operation of analyzing the difference in variances comprises performing an f-test based on each standard deviation of the background intensity and the foreground intensity.

3. The method of claim 1, wherein the operation of verifying the means comprises performing a pooled t-test or a non-pooled t-test, based on the difference in variances.

4. The method of claim 1, wherein the operation of verifying the means comprises increasing degrees of freedom if the difference in variances is high.

5. The method of claim 1, wherein the operation of judging an error spot is based on a p-value calculated from the results of the operation of verifying the significant difference of the means.

6. The method of claim 5, wherein in the operation of judging the error spot, a spot is judged as the error spot if the p-value is at least 0.05.

7. The method of claim 1, further comprising the operation of receiving resultant files generated from a quantifying process and parcing the resultant files to extract input data which are necessary in the operations of analyzing the difference in variances and verifying the means.

8. The method of claim 7, wherein the input data include a first mean and a first standard deviation of the background intensity, the number of pixels in the background, a second mean and a second standard deviation of the foreground intensity, and the number of pixels in the foreground.

9. A system for detecting an error spot, comprising:
a variance analysis part for analyzing a difference in variances for background intensity and a foreground intensity for each spot in a DNA chip;
a mean verifying part for verifying whether a mean of the background intensity and a mean of the foreground intensity are significantly different from each other, based on the difference in variances; and
an error spot judging part for judging an error spot based on the results of the verifying operation.

10. The system of claim 9, further comprising a data input part for receiving resultant files generated from a quantifying process and parcing the resultant files to extract input data which are necessary in the operations of analyzing the difference in variances and verifying the means.

11. The system of claim 10, wherein the input data include a first mean and a first standard deviation of the background intensity, the number of pixels in the background, a second mean and a second standard deviation of the foreground intensity, and the number of pixels in the foreground.

12. The system of claim 9, wherein the variance analysis part analyzes the difference in variances by performing an f-test based on each standard deviation of the background intensity and the foreground intensity.

13. The system of claim 9, wherein the mean verifying part verifies the significant difference of the means by performing a pooled t-test or a non-pooled t-test based on the difference in variances.

14. The system of claim 9, wherein the error spot judging part judges the error spot based on a p-value calculated from the results in the mean verifying part.

15. A computer-readable recording medium having recorded thereto a computer program for executing in a computer a method of detecting an error spot, the method comprising the operations of:
analyzing a difference in variances for a background intensity and a foreground intensity for each spot in a DNA chip;
verifying whether a mean of the background intensity and a mean of the foreground intensity are significantly different from each other based on the difference in variances; and
judging an error spot based on the results of the verifying operation.

16. The method of claim 3, wherein the operation of verifying the means comprises increasing degrees of freedom if the difference in variances is high.
